(19) United States
(12) Patent Application Publication
(10) Pub. No.: US 2023/0190211 A1
(43) Pub. Date: Jun. 22, 2023

(54) PET System with Mechanical Movement of Rigid Detectors for Optimized Imaging

(71) Applicant: Sino Canada Health Institute Inc., Winnipeg (CA)

(72) Inventors: James Schellenberg, Winnipeg (CA); John Saunders, Winnipeg (CA)

(21) Appl. No.: 17/922,538

(22) PCT Filed: Jul. 15, 2021
(86) PCT No.: PCT/CA2021/050978

§ 371 Date: Oct. 31, 2022

Related U.S. Application Data
(60) Provisional application No. 63/056,796, filed on Jul. 27, 2020.

(51) Int. Cl. A61B 6/03; A61B 6/04; G01T 1/29

(52) U.S. Cl. CPC A61B 6/037; A61B 6/0407; G01T 1/2985

(57) ABSTRACT

Described herein is a positron emission tomography (PET) detector array that has a plurality of detector elements, with each detector element mounted on a resilient, rigid support surface. A detector element movement system is connected to each rigid support surface, so that each detector element can be moved independently. This allows for the relative position of the individual detectors to be changed during imaging so that the imaging window can be increased or decreased during imaging, thereby allowing for improved, better resolution imaging of smaller volumes if desired.

### PRIOR APPLICATION INFORMATION

The instant application claims the benefit of U.S. Provisional Patent Application Ser. No. 63/056,798, filed Jul. 27, 2021 and titled “PET SYSTEM WITH MECHANICAL MOVEMENT OF RIGID DETECTORS FOR OPTIMIZED IMAGING”, the entire contents of which are incorporated herein by reference in their entirety for all purposes.

### BACKGROUND OF THE INVENTION

For human brain imaging using combined MRI and PET technologies and particularly for diagnostic, staging, pre-operative or therapy monitoring applications, a patient typically lies face-up on a bed and the bed and patient are moved into position inside the MRI+PET system. Such an MRI system may typically have a cylindrical bore of diameter 50 cm, 60 cm, 70 cm or larger or may be a design using two flat plates oriented above and below the patient, such as the Hitachi Altaire. These systems range in field strength from 7 Tesla down to 0.55 Tesla or lower. Such a PET system may typically either be built into and integrated with the MRI system, such as is the case for Siemens Biograph mMR systems or the PET system may be a retrofit insert PET system.

For brain imaging in which both PET and MRI are done at the same time, the optimum system would have a PET system and MRI coil system quite close to the patient head to allow for the best in sensitivity and spatial resolution.

For brain imaging applications in which the volume of interest is relatively small, an optimal system would allow the PET system to adjust its position to allow the highest possible resolution and sensitivity for the volume of interest.

In the Prior Art, PET imaging systems have been designed with various physical geometries which can be moved during the imaging session, such as for example:
Movable integrated scanner for surgical imaging applications, as shown in U.S. Pat. No. 8,295,905;
“Apparatus for Improving Image Resolution and Apparatus for Super-Resolution Photography Using Wobble Motion and Point Spread Function (PSF), in Positron Emission Tomography”, published as US20110268334A1, in which the PET cylinder wobbles;
“Method for acquiring pet image with ultra-high resolution using movement of pet device”, published as WO2013162172A1;
“Tomographic pet camera with adjustable diameter detector ring” U.S. Pat. No. 5,825,031A, in which the diameter of the PET cylinder could be changed; and
“Pet camera with individually rotatable detector modules and/or individually movable shielding sections”, U.S. Pat. No. 6,744,053.

These system designs allowed the detector elements to be moved in the radial direction to accommodate for object size, or in the axial direction before imaging is started in order to customize for a surgical procedure, or in a wobbling motion to optimize sampling of the lines of response.

However, what is needed is a PET system that can move axially and in curvature using rigid detector elements. These two motions are necessary to optimize imaging of smaller volumes within the brain.

### SUMMARY OF THE INVENTION

According to a first aspect of the invention, there is provided a positron emission tomography (PET) detector array comprising:
a plurality of detector elements, each detector element mounted on a resilient, rigid support surface, each detector element comprising a position indicator,
a detector element movement system connected to each respective rigid support surface, said detector element movement system being arranged to apply pressure individually to each respective rigid support surface for movement of each respective detector element connected to the corresponding respective rigid support surface, and
a detector element position detection system arranged to detect the position indicator of each respective detector element.

### BRIEF DESCRIPTION OF THE DRAWINGS

FIG. 1 shows an MRI 101, a docking station 102, a BrainPET Insert 103 and the patient lying on the MRI bed 104. The patient head 105 is lying on a headrest 106.

FIG. 2 shows some elements of the BrainPET insert, including the headholder 201, the MRI Receive Coil 202 with an approximate inner diameter of 265 mm, which fits inside the MRI Transmit Coil 203, which fits inside the PET Ring 204. The MRI Receive and Transmit coil together are called the MRI Coil. The MRI Coil and the PET Ring are designed to fit in one and only one way to ensure accuracy of the patient images.

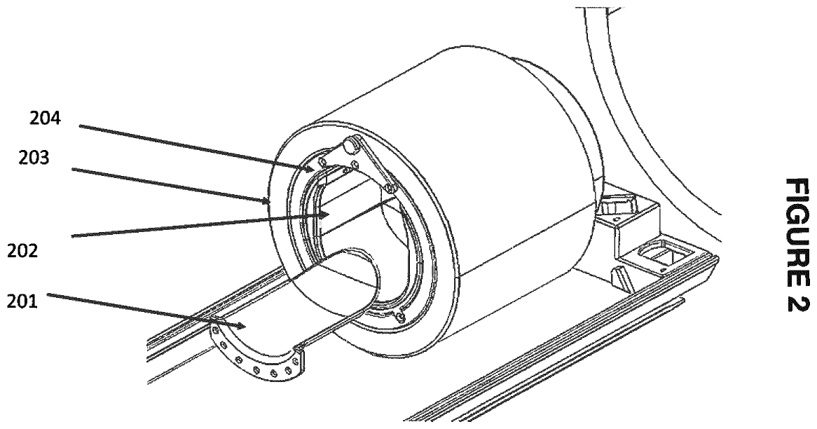

FIG. 3 shows a side view of the BrainPET Insert with a patient inserted. The Patient head 301 is on the headholder 305, and the head is encircled by the MRI Coil 302 and the PET Ring 303. Inside the PET Ring is shown PET Detector Elements 304. These PET Detector Elements are positioned axially and cylindrically around the patient head. For example, in some embodiments, there may be 3 PET Detector Elements in the axial direction and 16 sets of PET Detector Elements in the cylindrical direction, therefore allowing 48 PET Detector Elements to be arranged around the patient head in the PET ring design. As will be appreciated by one of skill in the art, different patient may have different head sizes due to biological differences between humans due to age, size, gender or other differences. For example, if each PET Detector Element is 32 mm long in the axial direction, and if there are 3 elements arranged axially, and if there is a small gap of 2 mm between each Detector Element, the maximum axial imaging field of view of the PET Ring will be 100 mm. The gap region between the Detector Elements does not cause an imaging gap of the patient because three-dimensional reconstruction is used in the system, and so there is still sampling of the entire patient head, as described below.

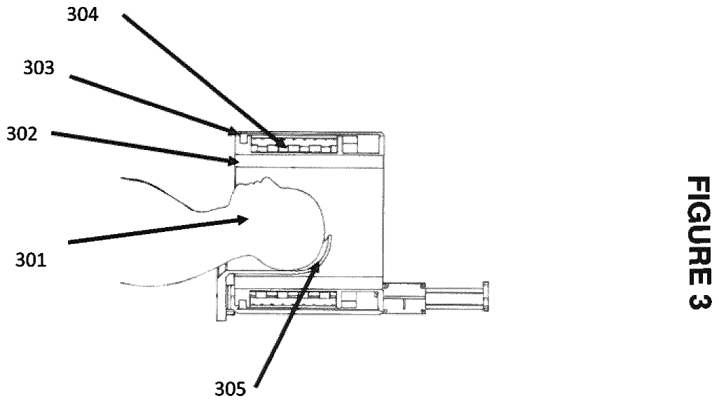

FIG. 4 shows a more detailed side view of one possible arrangement of the elements of the BrainPET Insert above the patient head. In these embodiments, first the patient head 407 of estimated axial extent 140 mm, then the plastic casing of the inner diameter of the insert 401 with axial length 260 mm, followed by an MRI coil 402 of axial length 240 mm, followed by PET detector elements 403 of total axial length 100 mm made up of three 32 mm detector elements with 2 mm gaps between, followed by a detector element movement system 404 of axial length 200 mm, followed by a detector element position measurement system 405 of axial length 200 mm, followed by an external plastic casing 406 of 260 mm axial length. This figure shows an embodiment with three PET Detector Elements arranged at one end of the axial direction. The MRI coil made up of transmit and receive portions will typically have a longer transmit coil of approximately 240 mm length and a receive coil made of multiple small loops arranged around the patient head.

FIG. 5 shows the PET sensitivity performance of the system shown in FIG. 4 under two different operating scenarios. One curve is the standard triangular sensitivity curve that would occur if the position of the PET Ring does not change during the imaging session. This is the position of the PET Ring in FIG. 4. In this case the PET Ring images the lower 100 mm of the patient head. In the second curve is shown the resulting sensitivity if the PET Ring is moved from the first position, in which it covers the lower 100 mm of the patient head, to a second position, in which it covers the upper 100 mm of the patient head. The movement from one position, as shown in FIG. 4, to the other, as shown in FIG. 6, would occur midway through the imaging session. We do not account for the decay of the radioactive agents in this simplified curve, or for the time it takes to move the PET Ring. This two position movement allows a total imaging region of 200 mm in the axial direction. The second curve is composed of two triangular sensitivity curves centered at 50 mm and 150 mm respectively.

FIG. 6 shows the second axial position. In this embodiment, the three detector elements are positioned at the other end of the PET Ring, and still have the 2 mm spacings between the PET Detector Elements.

FIG. 7 shows a combined sensitivity curve of 6 triangular sections. Specifically, this curve represents an embodiment with a more complicated set of movements—6 positions along the axial direction. The sampling position midpoints of the 6 locations in this embodiment are, for example, 50 mm, 70 mm, 90 mm, 110 mm, 130 mm and 150 mm. . As with the other curves radioactive decay and time required for the detector movements are not included in the calculation.

FIG. 8 shows the PET Detector Elements, three of them with a 2 mm gap between them, in the middle of the 200 mm axial extent, as an alternative to the axial movement discussed above. in

FIG. 9 shows the PET Detector Elements spaced apart with a gap spacing larger than 2 mm as another alternative to the axial movement discussed above. This type of movement also allows a larger head of, for example, 160 mm to be imaged using a smaller PET ring with an axial extent of say 100 mm. The resulting PET sensitivity curve will be a composite of the triangular sensitivity curves that would be obtained if there was no movement between positions. The amount of time spent in the positions will affect the particular sensitivity measured along the full 200 mm axial extent. More detailed calculations for expected sensitivity would account for radioactive decay and for the movement time required by the PET Detector Elements.

FIG. 10 shows a sensitivity curve for the type of movement discussed above.

FIG. 11 shows a third type of movement of the PET Detector Elements in which the movement system provides axial curvature of the PET Detector Elements.

FIG. 12 shows the Elements of the BrainPET Imaging System.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a PET system that can move axially and in curvature using rigid detector elements. These two motions are sufficient to optimize imaging for smaller volumes within the brain such as for example the subthalamic nucleus (STN).

As will be appreciated by one of skill in the art, the advantage of moving of detector elements during imaging, that is, once information is known about the size and orientation of the object being imaged, is so that the PET imaging can be optimized, once specifics such as for example the region of interest within the object, the inherent tissue properties of the object, and the activity properties of the radioactive distribution within the object are known, as discussed herein. That is, if the detection elements can be moved during imaging, the effectiveness of the PET scanning can be maximized.

As will be appreciated by one of skill in the art, there are many possible methods for determining the optimum or preferred position to achieve specific imaging goals and/or reduce errors. For example, the system may be used to provide PET system movement prior to having the patient in the field of view, for example by considering the patient head size and orientation in the headholder, and then estimating the region of interest size and location within the patient head. The PET system would then be moved in axial and curvature directions to optimize imaging of the predicted region.

In practical terms, it may be impossible to have a large number of calibration tables for the PET system, and so the system may choose the best PET system orientation from a standard set of premeasured calibration tables.

It is understood that the PET attenuation caused by the head will need to be calculated for the different PET system orientations. In this case, a lookup table for the calibration procedures may be used.

A different approach would have a set of calibration estimation calculations which can be done for different positions of the PET system, and these calibration estimation calculations would be able to estimate the calibration parameters of a large number of positions from the measured calibration parameters of a smaller number of positions.

However, in all cases, the ability to adjust the positioning of the detector elements during imaging as discussed herein will improve the usefulness and sensitivity of PET scanning, as discussed herein.

For example, in some cases, a large object may need imaging by a smaller set of PET Detectors. A patient head, for example, may be 160 mm in axial extent but for price and cost reasons, the axial extent of the PET Detector Elements may only be 100 mm. In this case, the movements that are discussed herein can be used to extend and optimize the imaging volume.

For example, a BrainPET insert optimized for imaging the brain is arranged to be inserted into an MRI to allow simultaneous PET+MRI imaging. As different sizes of human heads will be inserted into the BrainPET, if the object of interest is a relatively small volume of the brain, such as for example the Sub Thalamic Nucleus (STN), the location of the STN will be different for at least every different head size being imaged. If for example the STN is the object of most interest within the brain, it would be useful to be able to optimize the PET system to image the STN with the best possible spatial resolution and/or sensitivity. The STN is used as an example herein because the STN is relatively small, about the size of 7 mm length and width. The STN is important as a target for some Deep Brain Stimulation (DBS) procedures. DBS is a technique that allows electronics to be placed in the brain to affect the electrical behaviour of the brain. In the same way that cardiac pacemakers can be used to adjust and stabilize the operation of the heart, neuro electronics may be useful to regulate electrical signals to control Parkinson tremors and other movement and dystonia. Other parts of the brain may also be of interest for other disease applications. In these systems, part of the electronics is typically left in the brain on a multi-year basis, electrode wires are connected out of the head to a body mounted receiver, and the system is controlled using other methods.

To optimize performance of a BrainPET used for this type of small volume imaging, a small but moveable set of detector elements can be used, as discussed herein. For example, the PET field of view may be concentrated to image a small region. In addition, even if the PET field of view is quite large, it is sometimes useful to physically adjust and orient the PET system so that the emitted radioactivity can be detected in an optimized way.

This physical orientation and adjustment of the detector elements can be done before the patient is inserted into the PET/MRI system, after an MRI image is acquired, or during the acquisition of the PET/MRI images.

Prior to patient insertion, the head size and region of interest can be measured and calculated and the optimal BrainPET element position can be calculated, as discussed herein. After patient insertion, an MRI image can be quickly taken to allow better estimation of the location of the region of interest, and the PET system would then be positioned in the best position for the head being examined. During PET/MRI operation, optimization could be done based on detected signals from the patient. In addition, in some embodiments, it may be of interest to track the location of radioactive sensors in the brain which are attached to DBS electrode insertion systems. In addition, in some embodiments, it may be of interest to track the location of radioactive distributions in the brain during an imaging session, with the optimal tracking of the radioactive distribution being performed using a flexible PET system.

As discussed herein, flexible scintillator material and flexible PCB boards and readout board systems are not being used in this design, and so a rigid detector element is assumed which consists of a scintillator of fixed size, SiPM pixels of fixed size, and optical gels and glue and reflective materials sufficient to create the desired integrated and rigid detector element. These detector elements are rigid in themselves and provide a rigid flat surface area which can be oriented towards or away from a given location. Each detector element has a number of electronic signal lines coming from it, and so in some embodiments there is a tradeoff in this design between having many detector elements to achieve greater flexibility in curvature, or fewer detector elements leading to decreased number of signal lines and decreased flexibility of curvature. While not wishing to be bound to a particular theory or hypothesis, the inventors believe this flexibility of curvature may be important in minimizing the creation of artifacts during the reconstruction process. In current PET systems, the gap between detector elements is carefully designed so that the reconstruction software can effectively account for the gap during software operation. In the flexible system described herein, this gap filling algorithm approach will be complicated because of the flexibility of the positioning of the detector elements; however, this complexity will be greatest when using a smaller number of larger detectors.

According to an aspect of the invention, there is provided a positron emission tomography (PET) detector array comprising:
a plurality of detector elements, each detector element mounted on a resilient, rigid support surface, each detector element comprising a position indicator,
a detector element movement system connected to each respective rigid support surface, said detector element movement system being arranged to apply pressure individually to each respective rigid support surface for movement of each respective detector element connected to the corresponding respective rigid support surface, and
a detector element position detection system arranged to detect the position indicator of each respective detector element.

The detector element movement system may be arranged to move a respective detector element such that the position of the respective detector element is moved in an axial direction.

The detector element movement system may be arranged to move a respective detector element such that a gap between the respective detector element and an adjacent detector element is increased or decreased.

The detector element movement system may be arranged to move a respective detector element such that axial curvature of the respective detector element is increased or decreased.

For example, the detector element movement system may be arranged to move each respective detector element up to 100 mm in an axial direction and/or up to 20 mm in a radial direction.

The detector element position detection system may detect the position indicator of a respective detector element to within 1 mm.

The detector element movement system may apply pressure to the rigid support surfaces by at least one air bladder.

In some embodiments, the detector elements are arranged in a cylindrical array.

For example, the detector elements may be arranged in a cylindrical array of two or more approximately equidistantly spaced columns.

In some embodiments, there are 16 approximately equidistantly spaced columns.

In some embodiments, there are two or more equidistantly spaced detectors in each respective one column.

For example, there may be 3 approximately equidistantly spaced detectors in each respective one column.

In some embodiments, each respective one column is mounted to a respective one rigid support surface.

For example, there may be a separate respective air bladder attached to each respective rigid support surface or there may be one common air bladder arranged such that individual cells of the one common air bladder control movement of a respective rigid support surface.

Alternatively, the detector element movement system may apply pressure to the respective rigid support surfaces by an articulated arm or by a geared system.

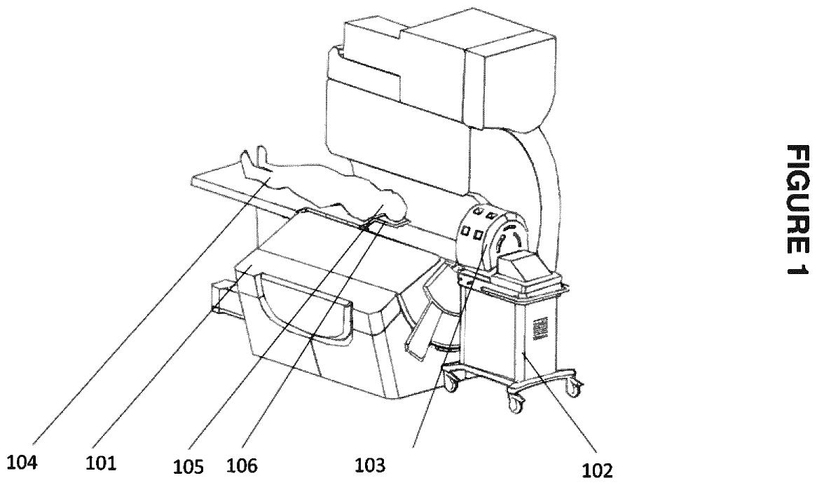

Shown schematically in FIG. 1 is a BrainPET Imaging system which can be inserted into an MRI system, comprising a previously installed MRI 101; a docking station 102 that contains analog to digital conversion electronics; a BrainPET Insert 103 that slides into the MRI, and which is connected to the docking station by cabling; and a patient 104. Not shown in the figure is the data acquisition, reconstruction and analysis workstation and software, which can be installed in the MR equipment room or control room in some embodiments. The connection between the docking station and the workstation is fiber. Also not shown is the control workstation with control and measurement software to allow position movement and position measurement of internal elements of the BrainPET Insert.

In one embodiment of the invention, the BrainPET Insert comprises:
1. one integrated MRI coil,
2. a PET ring with detector elements,
3. the end effectors of a detector element movement system, and
4. the end effectors of a position detection system.

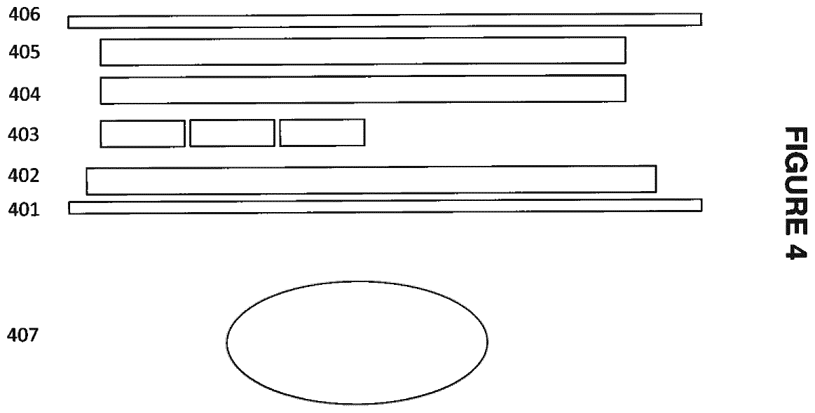

FIG. 4 shows the sequence of elements of the BrainPET Insert, with first the plastic casing of the inner diameter of the insert 401, followed by a MRI brain imaging coil 402, followed by PET detector elements 403, followed by a detector element movement system 404, followed by a detector element position measurement system 405, followed by an external plastic casing 406. These items encircle the patient head 407. Electrical wiring for this system is not shown. Cooling systems for this type of PET insert design may or may not be needed. It is further noted that suitable methods of cooling are known in the art and may be incorporated if needed or as needed.

The plastic inner cover 401 is thick enough to allow rigidity and support for the interior elements. In this exemplary embodiment, approximately 10 mm is allocated for this. As used herein, “approximately” refers to a value that is plus or minus 10% of the base value. As such, in this example, “approximately 10 mm” means “approximately 9-11 mm”.

The MRI Coil 402 is approximately 25 mm thick or less and can be of a transmit and receive variety. The field of view of the MRI coil will be approximately 200 mm for transmit and up to approximately 200 mm for receive, and the same copper circuits may be used for both transmit and receive or separate transmit and receive coils can be used. The MRI coil system encircles the patient head in a circumference.

In the exemplary embodiment, the PET system comprises 48 detector elements (DE) that form a cylinder around the inner volume where the patient head will be located, with each DE mounted on a DE movement system 404. In this exemplary embodiment, 3 DEs are in one axial column and there are 16 columns around the circumference of the PET ring. These DEs move as discussed below, and it is assumed that 50 mm is allocated for PET ring thickness.

The DEs consist of scintillator blocks, readout board elements, optical glue or gel, and reflective tapes and optical methods to allow readout of the scintillation events. In this example, each PET detector element is 50 mm wide and 32 mm long in the axial direction, with 2 mm gap between detector elements in the axial direction, with the 3 DEs arranged along the access to allow for 100 mm of axial field of view. In this embodiment, the 16 columns can be evenly spaced around the circumference to allow for a transaxial PET field of view of approximately 250 mm. As will be appreciated by one of skill in the art, other suitable arrangements may be desirable for certain purposes and are within the scope of the invention. The size of 250 mm diameter is chosen to allow 95% of human heads to fit within the imaging volume.

If the detector elements are 50 mm wide and there are 5 to 10 mm between detector elements in the cylindrical direction, one can use 16 columns of DEs to wrap a complete circumference around the patient head. There are 3 DEs per column, thus requiring 48 DE elements in total. As will be known by those of skill in the art, the space between the DEs is used for manufacturing and movement tolerances.

The PET Detector elements 403 can be up to 25 or 30 mm thick, depending on the thickness of the scintillator, the type of scintillator used, and the type of readout electronics and detection ASIC electronics used in the detector element. In the exemplary embodiment, the PET detector elements 403 are of a scintillator type LYSO, are designed to be 20 mm thick, have typical dimensions of 33 mm×48 mm, and can have a weight estimated at 315 grams. This weight is made up of 215 grams for the scintillator, with density approximately 7.1 grams per cubic cm, and 100 grams for the readout board and electronics. The scintillator width 48 mm is slightly smaller than the total detector element width 50 mm to allow for mounting room.

The detector element position movement system 404 is an MR compatible system that can move detector elements in the axial direction and radial direction. In the axial direction, a typical range of motion might be approximately 100 mm, whereas in the radial direction the range of motion is typically approximately 20 mm. The DE position movement system is assumed to be of approximately 20 mm thickness.

One method for DE movement may include air filled bladders that expand or contract to move the DEs. Alternatively, in other embodiments, the DE movement system may be an articulated arm mechanism or a geared system mounted on the back of the DE.

The DE position measurement system relays coordinate information back to the reconstruction software to ensure that the correct positioning of each scintillator crystal is being properly recorded for image reconstruction usage, as discussed herein.

The detector element position measurement system 405 will be able to measure the location of the detector elements to an accuracy of, for example, 1 mm. The DE position measurement system is assumed to be of thickness 5 mm and extends, for example, from 225 mm to 230 mm from the middle of the bore.

The level of position measurement accuracy is related to the scintillator design. In a typical PET system, the scintillator blocks are constructed of LYSO crystals of size 2 mm×2 mm×20 mm, and the location of the crystal needs to be known or calibrated in real space in order to perform reconstruction and gain good spatial resolution. For 2 mm crystal sizes, it is assumed that 1 mm of position measurement accuracy would be sufficient. However, there are also scintillator designs that have a layered approach, in which smaller crystals are stacked in an offset fashion to achieve increased spatial resolution.

One embodiment of a position measurement system 404 that is commercially available and which can be integrated with this system is the Endoscout system from Robin Medical. This system uses sensors in the shape of flat surfaces, cubes or cylinders that allow the measurement of position inside the MRI while the sequences are running. If such a system is used for system 405, this allows the position measurement system to be separated from the position movement system.

Another suitable example of a position measurement system 404 is a fiducialed-based system in which fiducials are attached to the detector elements, and an MRI coil is positioned, so that the MRI coil measures the fiducial locations during the imaging sequence or during specialized hunt sequences for locating the fiducials.

In another embodiment, the DE position measurement system may be a pre-programmed location map for specific orientations of the DEs.

The DE movement system allows three types of movements to be made, which are:
1. Axial Shift
2. Axial Gap
3. Axial Curvature

### Axial Shift

In some embodiments, an MRI coil will have a field of view of for example approximately 200 mm, and a PET ring will be smaller, with only an axial field of view of approximately 100 mm. The PET ring can be slid forward or backward in the PET Insert to allow it to image different parts of the brain. Three of the expensive components in these systems are the pixels, data acquisition electronics and scintillators, and so any reduction in the number and amount of these detector elements will reduce the cost of the system.

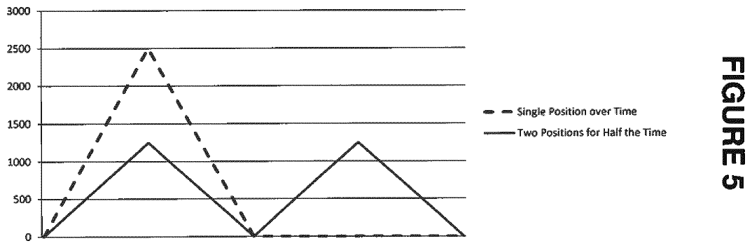

FIG. 5 illustrates the sensitivity performance that can be expected over a 200 mm axial length from two methods of use of a 100 mm long PET ring. If the PET Ring Detector Elements are placed at one end of the 200 mm axial field of view, then the commonly occurring triangular PET sensitivity is achieved. In this case, the normal sensitivity curve of a triangle over the first 100 mm, but the total 200 mm axial length of interest is not sampled. Alternatively, if the PET ring Detector Elements are located first at one end of the 200 mm Axial length, but then are shifted to the other end of the 200 mm axial length at the midway point of the imaging session, the combination of two triangular sensitivity patterns are found. The entire 200 mm axial length is sampled in this case.

In this simple example, the effect of radioactive decay is not included, and so it is assumed that the source continues to emit the same level of radioactivity over the session. As will be appreciated by one of skill in the art, more detailed protocols allow for the movement of the PET ring to the second location slightly earlier if the goal is to equalize the counts for both positions.

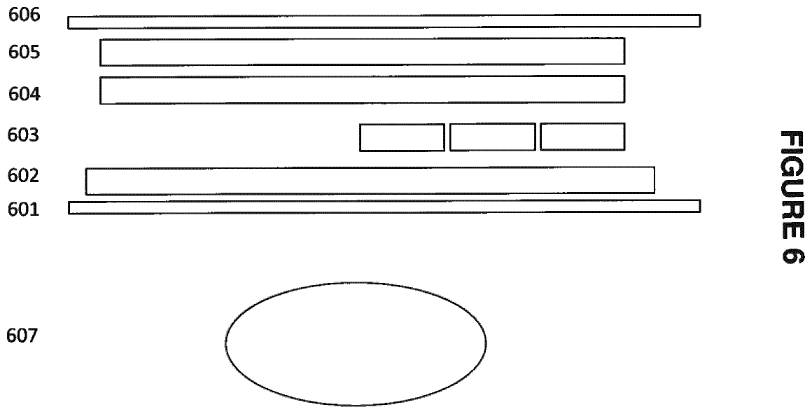

FIG. 6 shows the second axial position related to the Two Positions curve of FIG. 5.

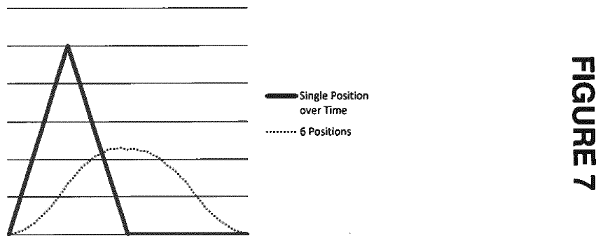

This example only uses axial shift, and so the gap distance between the PET rings is maintained at the same amount, for example, about 2 mm, throughout the imaging session. With this gap amount, various other sampling protocols could be used. For example, if a more evenly distributed sensitivity curve is desired, 6 axial positions could be used. The sensitivity that is achieved in this case is shown in FIG. 7 which shows sampling a 200 mm object using a 100 mm PET Ring with 6 axial locations.

If 6 axial shift positions are used, with the edge of the 100 mm PET ring being at the locations 0, 20, 40, 60, 80, 100 mm then the sensitivity curve will be smoothed out and will appear as shown in FIG. 6. The triangular curve is achieved if the 100 mm PET ring is fixed at one end of the 200 mm region of interest, but the smoothed curve is achieved if the PET ring is slid in the axial direction. The area under the two curves is the same—there is no difference in the number of counts in this theoretical example. In practice, this theoretical example curve may not be achieved if the sliding motion is so slow that it affects a significant portion of the imaging session. For example, if the imaging is of the human brain, and the imaging session is 30 minutes long, and if the sliding motion requires 2 minutes to move the distance of 20 mm, then a total of 10 minutes would be used to move the PET ring, and this would probably cause significant blurring of the PET image unless motion compensation methods were used.

This axial shift approach to imaging is of particular interest because the cost of the PET imaging system is strongly related to the number of detector elements that are used in the design. If it is possible to use fewer detector elements and to move them around the patient to achieve a result, then the price of the PET system may be reduced. Of course, the system used to move the PET detectors must cost less than the PET detector elements that they are saving. In PET systems today, the scintillator, the pixels and the data acquisition electronics are the expensive parts of the design, and all three of these are reduced using this sliding axial approach, as discussed herein.

In addition, there is a strong trend today to use AI techniques, such as deep learning, machine learning, artificial intelligence and related techniques, to reduce the imaging dose that is required. Incorporating AI approaches that require fewer counts to form an image can be advantageously combined with this sliding axial technique, as discussed herein.

Notice in the figure that the highest number of counts is achieved if the PET system is held in position 1 only. The tradeoff is that the axial region that can be imaged is only 100 mm in length. Using the 6 position method, the entire 200 mm of the region of interest can be measured but the number of counts along the axis is lower. However, if AI methods can be used, this lower number of counts will not matter. Radioactive decay is also not considered in these calculations.

### Axial Gap

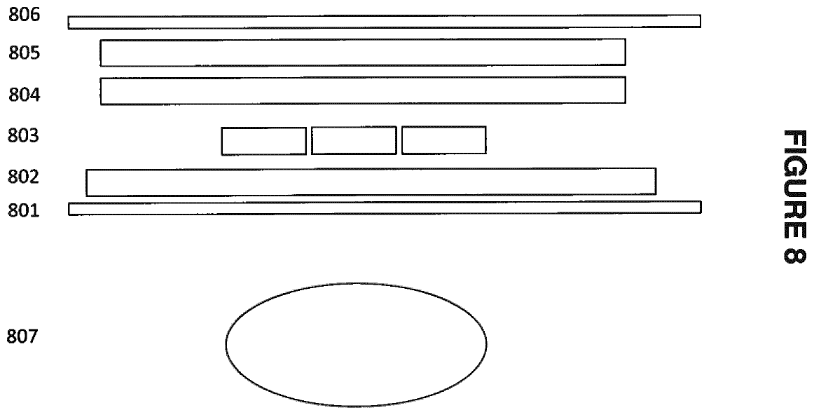

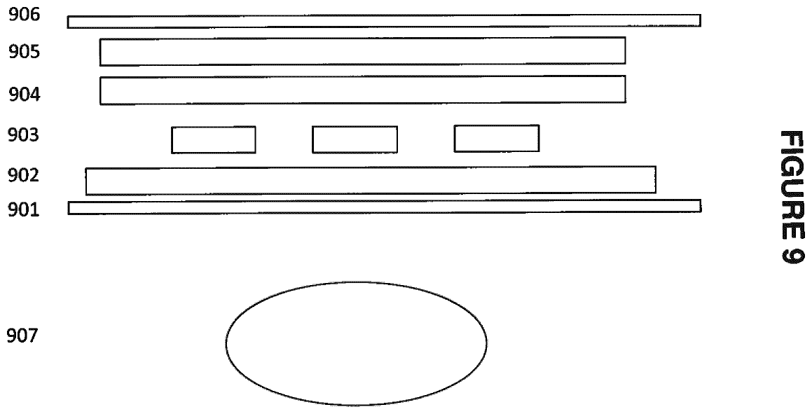

In other embodiments, the PET ring will allow the gap spacing between detector elements to be varied. This allows smoothing of the sensitivity and optimization of the imaging. The total axial length can be larger than 100 mm in this case, but the axial gap will cause the sensitivity to flatten out in this location. The gap sensitivity will not go to 0, and so information about the PET emissions from this volume between the gaps is still available. This technique can be used to reduce cost but maintain sensitivity over a wider axial extent. FIG. 8 shows one example of imaging with a certain gap configuration, and FIG. 9 shows a different configuration with increased gap amount. In FIG. 8, the axial sensitivity would be a 100 mm long triangular pattern centered in the middle of the 200 mm total axial extent.

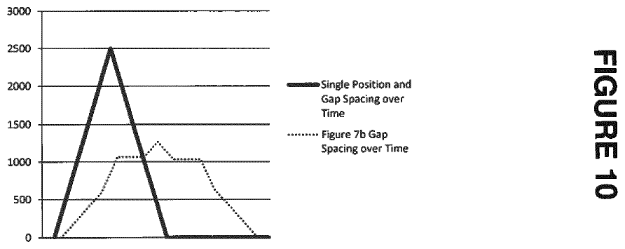

FIG. 10 shows the resulting sensitivity profiles that are achieved for these two positionings of the detector elements. The normal triangular pattern is achieved in the first case, but in the second, the sensitivity in the gap is filled in via the 3D reconstruction that is done. This gap sensitivity is directly related to the number of lines of response that occur through the gap volume.

This axial gap approach requires a more sophisticated gap filling algorithm than the axial shift approach but achieves the same end. This axial gap approach is an alternative way of moving the detector elements. Specifically, whereas the axial shift approach requires the entire 100 mm of detector elements to move, the axial gap approach will require a movement system that only moves the outer two detector elements while leaving the middle one unmoved. This type of movement may be useful in some designs. These detector elements may be quite heavy. For example, for a typical BrainPET design, the detector element may be 40 mm×50 mm, with a LYSO scintillator thickness of 20 mm. LYSO has a density of 7.1 grams per cm³, and so for a BrainPET detector element of 40 cm³ this weighs 284 grams. As will be appreciated by one of skill in the art, axial gap or axial shift may be the preferred implementation, depending on the desired cost, complexity and performance of the movement system.

### Axial Curvature

In some embodiments, the PET ring has its curvature modified, as if it is focusing on a region of interest in the middle of the PET cylinder. The effect of pointing all the crystal elements at a centralized location in the bore is that the parallax effect which causes DOI depth of interaction effects is reduced and/or eliminated, leading to improved spatial resolution performance. The curvature may also allow a change in reconstruction method to be done. As will be appreciated by one of skill in the art, the sensitivity of the device is not affected, or is affected minimally, because the scintillator blocks still extend close to the full radial extent.

The parallax problem occurs because the object to be reconstructed is not directly in line with the detector elements. This off-axis location means that the effective scintillator thickness looks different depending on the relative orientation of the PET detector crystal and the object of interest, leading to reconstruction blurring of the object. This blurring will be different in the axial, radial and tangential directions. However, if all detector elements point at the region of interest, then the image can be reconstructed without any blurring, leading to a crisper image.

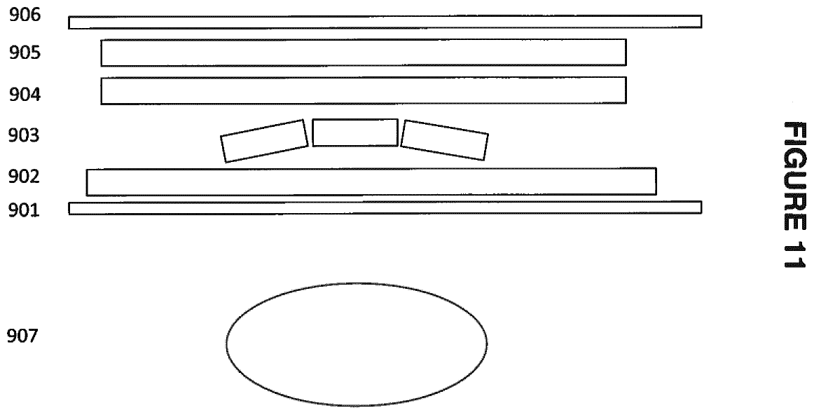

FIG. 11 shows a side view of the PET Ring with detector elements in a curved orientation. The detector elements themselves are rigid and with flat surfaces, but the lifting of the middle detector element and the lifting of the sides of the other elements create a curved total view.

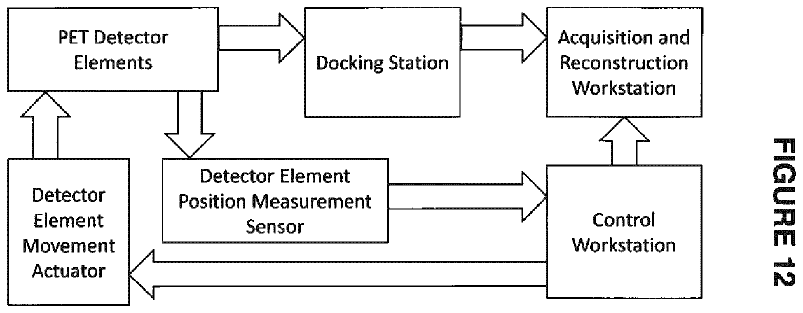

FIG. 12 shows the Elements of the BrainPET Imaging System. These elements are implemented in hardware, firmware and software. The PET Ring transmits raw voltage data to the docking station, which converts the voltage data to ListMode values that indicate where and when scintillator events have occurred. This singles data is passed to the acquisition and reconstruction workstation to allow coincident processing, image reconstruction, and other algorithms to be done. Once the workstation finishes creating DICOM image files, these files can be transferred into the network, typically into the PACS system. In those cases where MRI DICOM files are also available, the MRI and PET DiCOM files can be used together to create simultaneous PET-MRI images.

The control workstation provides control and monitoring of the DE movement and position measurement. The control workstation may be a separate workstation installed in a different location than the MRI room, such as behind a filter panel, or it may be located inside the same chassis that houses the acquisition and reconstruction workstation or it may be contained in the docking station. The control workstation passes movement commands to the movement actuator, which then moves the detector elements inside the insert. The position measurement sensors then relay position data to the control workstation. Notice that implicit in this setup is that the movement system does not necessarily know where the detector elements are and relies on the position measurement system to determine whether the detector elements are in the right spot.

Between the detector element position measurement system and the docking station, there may be a timing interface. This timing interface is used in those applications and system designs in which the time at which the various detector elements are in the various positions needs to be inputted as a gating signal to the reconstruction software.

In the simplest version of this system, there will be predetermined locations for the detector elements, with predetermined calibration tables and other system matrix calculations and tables, as discussed herein.

For these types of PET systems, if axial curvature is not used then the PET Ring can be made thinner than with axial curvature. As will be appreciated by one of skill in the art, different PET Insert designs will be suitable for different applications and uses but are within the scope of the invention.

While the preferred embodiments of the invention have been described above, it will be recognized and understood that various modifications may be made therein, and the appended claims are intended to cover all such modifications which may fall within the spirit and scope of the invention.

1. A positron emission tomography (PET) detector array comprising:
a plurality of detector elements, each detector element mounted on a resilient, rigid support surface, each detector element comprising a position indicator,
a detector element movement system connected to each respective rigid support surface, said detector element movement system being arranged to apply pressure individually to each respective rigid support surface for movement of each respective detector element connected to the corresponding respective rigid support surface, and
a detector element position detection system arranged to detect the position indicator of each respective detector element.

2. The PET detector array according to claim 1 wherein the detector element movement system is arranged to move a respective detector element such that the position of the respective detector element is moved in an axial direction.

3. The PET detector array according to claim 1 wherein the detector element movement system is arranged to move a respective detector element such that a gap between the respective detector element and an adjacent detector element is increased or decreased.

4. The PET detector array according to claim 1 wherein the detector element movement system is arranged to move a respective detector element such that axial curvature of the respective detector element is increased or decreased.

5. The PET detector array according to claim 1 wherein the detector element position detection system detects the position indicator of a respective detector element to within 1 mm.

6. The PET detector array according to claim 1 wherein the detector element movement system applies pressure to the rigid support surfaces by at least one air bladder.

7. The PET detector array according to claim 1 wherein the detector element movement system applies pressure to the respective rigid support surfaces by an articulated arm.

8. The PET detector array according to claim 1 wherein the detector element movement system applies pressure to the respective rigid support surfaces by a geared system.

9. The PET detector array according to claim 1 wherein the detector elements are arranged in a cylindrical array.

10. The PET detector array according to claim 1 wherein the detector elements are arranged in a cylindrical array of two or more approximately equidistantly spaced columns.

11. The PET detector array according to claim 10 wherein there are 16 approximately equidistantly spaced columns.

12. The PET detector array according to claim 10 wherein there are two or more equidistantly spaced detectors in each respective one column.

13. The PET detector array according to claim 12 wherein there are 3 approximately equidistantly spaced detectors in each respective one column.

14. The PET detector array according to claim 10 wherein each respective one column is mounted to a respective one rigid support surface.
